(19) United States
(12) Patent Application Publication
(10) Pub. No.: US 2026/0224278 A1
(43) Pub. Date: Aug. 6, 2026

(54) ELECTRONIC LATCH FOR ELECTROSURGICAL INSTRUMENT

(71) Applicant: Cilag GmbH International, Zug (CH)

(72) Inventor: Brendan J. Oberkircher, Cincinnati, OH (US)

(21) Appl. No.: 19/044,771

(22) Filed: Feb. 4, 2025

(51) Int. Cl. A61B 18/14; A61B 17/29; A61B 18/00; A61B 34/00

(52) U.S. Cl. CPC A61B 18/1445; A61B 34/25; A61B 2017/2925; A61B 2017/2946; A61B 2018/00702

(57) ABSTRACT

A surgical instrument includes a handle, an end effector, a trigger, a lock, a sensor, and a processor. The trigger is moveable relative to the handle between a first position and a second position; and from the second position to a third position. The trigger is configured to actuate the end effector upon a movement of the trigger from the first position to the second position. The lock in a locked configuration is configured to prevent the trigger from moving from the second position to the second position. The processor is configured to receive a mode selected from a plurality of available modes. The processor is further configured to provide a response to detecting that the trigger is in the third position, the response being based on which mode from the plurality of available modes has been selected.

### BACKGROUND

A variety of surgical instruments include a tissue cutting element and one or more elements that transmit radio frequency (RF) energy to tissue (e.g., to coagulate or seal the tissue). An example of such an electrosurgical instrument is the ENSEAL® Tissue Sealing Device by Ethicon Endo-Surgery, Inc., of Cincinnati, Ohio. Further examples of such electrosurgical instruments and related concepts are disclosed in U.S. Pat. No. 6,500,176 entitled “Electrosurgical Systems and Techniques for Sealing Tissue,” issued Dec. 31, 2002, the disclosure of which is incorporated by reference herein, in its entirety; U.S. Pat. No. 9,526,565, entitled “Electrosurgical Devices,” issued Dec. 27, 2016, the disclosure of which is incorporated by reference herein, in its entirety; U.S. Pat. No. 11,857,247, entitled “Jaw for Surgical Instrument End Effector,” issued Jan. 2, 2024, the disclosure of which is incorporated by reference herein, in its entirety; and U.S. Pub. No. 2022/0008120, entitled “Electrosurgical Instrument with Floating Jaw Component,” published Jan. 13, 2022, the disclosure of which is incorporated by reference herein in its entirety.

An electrosurgical instrument may be powered by an external generator. Examples of such generators are disclosed in U.S. Pat. No. 8,986,302, entitled “Surgical Generator for Ultrasonic and Electrosurgical Devices,” issued Mar. 24, 2015, the disclosure of which is incorporated by reference herein, in its entirety.

Some electrosurgical instruments may be supported and driven by a robotic surgical system. Examples of robotically controlled electrosurgical instruments are disclosed in U.S. Pat. No. 11,576,738, entitled “Systems and Instruments for Tissue Sealing,” issued Feb. 14, 2023, the disclosure of which is incorporated by reference herein in its entirety; and U.S. Pub. No. 2022/0338891, entitled “Systems for Setting Jaw Gap in Surgical Tool End Effectors,” published Oct. 27, 2022, the disclosure of which is incorporated by reference herein in its entirety.

Insomuch as a variety of surgical instruments have been in use, so too have been a variety of modes of operation for these surgical instruments. Over time, operators may deviate from one another on preferences of which mode is preferred, leading to multiple instruments having differentiated features and components to accommodate a particular mode. Having multiple surgical instruments with different modes of operation could increase costs to hospitals and could result in operators not receiving a surgical instrument with their preferred mode of operation. Further, different modes of operations may be beneficial for different aspects of a single procedure such that multiple similarly indicated surgical instruments are needed for a single procedure.

While a variety of surgical instruments have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of a first example of an electrosurgical instrument;

FIG. 2 depicts a perspective view of an articulation assembly and end effector of the electrosurgical instrument of FIG. 1, with the end effector in an open configuration;

FIG. 3 depicts an exploded perspective view of the articulation assembly and end effector of FIG. 2;

FIG. 4 depicts a perspective view of the end effector of FIG. 2, with the end effector in an open configuration;

FIG. 5 depicts an exploded perspective view of the end effector of FIG. 2;

FIG. 6 depicts a schematic view of a second example electrosurgical instrument, with additional components including a processor shown in schematic form;

FIG. 7 depicts a schematic view of a pistol grip and trigger of the electrosurgical instrument of FIG. 6;

FIG. 8 depicts a graphical user interface that may be driven by the processor of FIG. 6, showing first, second, and third selectable modes;

FIG. 9 depicts the graphical user interface of FIG. 6, showing the third selectable mode of FIG. 8 selected;

FIG. 10 depicts the graphical user interface of FIG. 6, showing the first selectable mode of FIG. 8 selected;

FIG. 11A depicts a schematic view of the electrosurgical instrument of FIG. 6, showing a first procedural step of the first selectable mode of FIG. 8;

FIG. 11B depicts a schematic view of the electrosurgical instrument of FIG. 6, showing a second procedural step of the first selectable mode of FIG. 8;

FIG. 11C depicts a schematic view of the electrosurgical instrument of FIG. 6, showing a third procedural step of the first selectable mode of FIG. 8;

FIG. 11D depicts a schematic view of the electrosurgical instrument of FIG. 6, showing a fourth procedural step of the first selectable mode of FIG. 8;

FIG. 11E depicts a schematic view of the electrosurgical instrument of FIG. 6, showing a fifth procedural step of the first selectable mode of FIG. 8;

FIG. 11F depicts a schematic view of the electrosurgical instrument of FIG. 6, showing a sixth procedural step of the first selectable mode of FIG. 8;

FIG. 11G depicts a schematic view of the electrosurgical instrument of FIG. 6, showing a seventh procedural step of the first selectable mode of FIG. 8;

FIG. 11H depicts a schematic view of the electrosurgical instrument of FIG. 6, showing an eight procedural step of the first selectable mode of FIG. 8;

FIG. 11I depicts a schematic view of the electrosurgical instrument of FIG. 6, showing a ninth procedural step of the first selectable mode of FIG. 8;

FIG. 12 depicts the graphical user interface of FIG. 8, showing the second selectable mode of FIG. 8 selected;

FIG. 13A depicts a schematic view of the electrosurgical instrument of FIG. 6, showing a first procedural step of the second selectable mode of FIG. 8;

FIG. 13B depicts a schematic view of the electrosurgical instrument of FIG. 6, showing a second procedural step of the second selectable mode of FIG. 8;

FIG. 13C depicts a schematic view of the electrosurgical instrument of FIG. 6, showing a third procedural step of the second selectable mode of FIG. 8;

FIG. 13D depicts a schematic view of the electrosurgical instrument of FIG. 6, showing a fourth procedural step of the second selectable mode of FIG. 8;

FIG. 14 depicts the graphical user interface of FIG. 8, showing the third selectable mode of FIG. 8 selected;

FIG. 15A depicts a schematic view of the electrosurgical instrument of FIG. 6, showing a first procedural step of the third selectable mode of FIG. 8;

FIG. 15B depicts a schematic view of the electrosurgical instrument of FIG. 6, showing a second procedural step of the third selectable mode of FIG. 8;

FIG. 15C depicts a schematic view of the electrosurgical instrument of FIG. 6, showing a third procedural step of the third selectable mode of FIG. 8; and

FIG. 15D depicts a schematic view of the electrosurgical instrument of FIG. 6, showing a fourth procedural step of the third selectable mode of FIG. 8.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

### I. First Example of an Electrosurgical Instrument

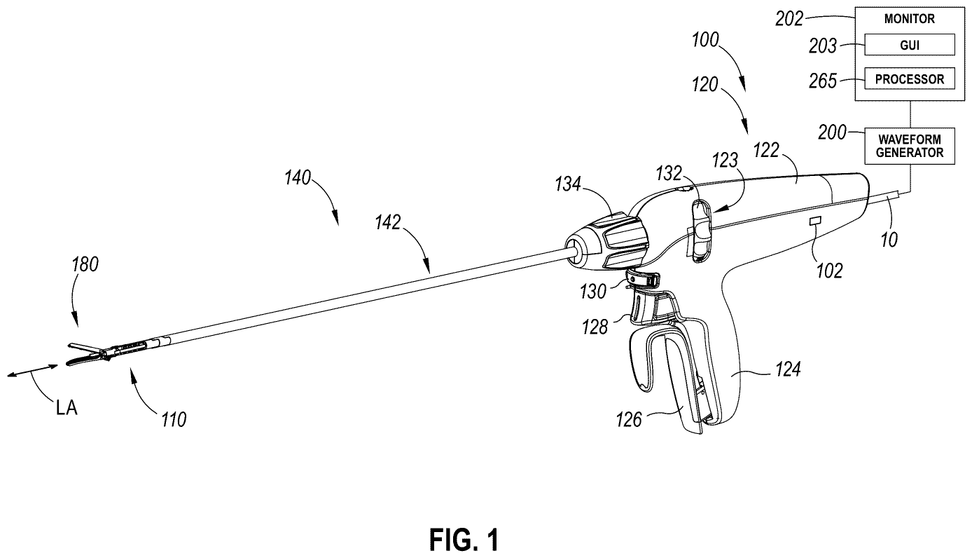

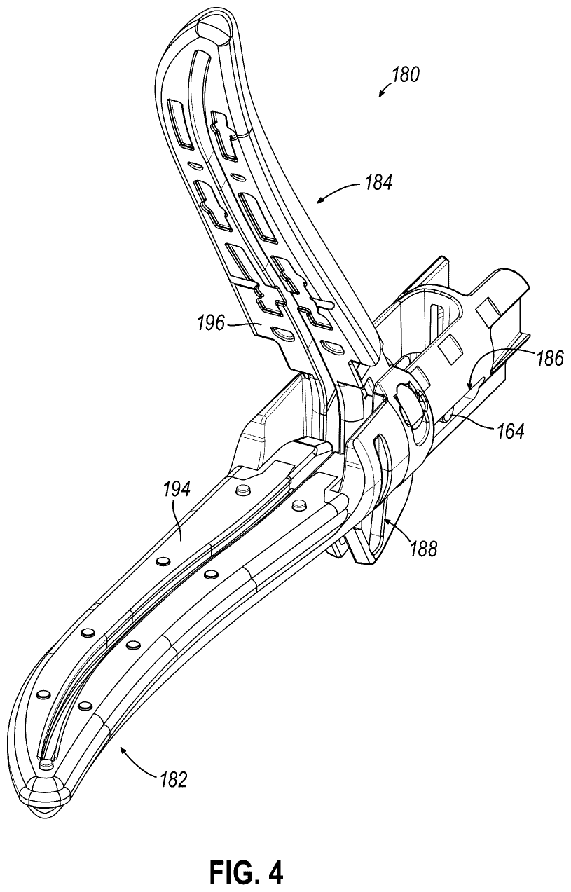

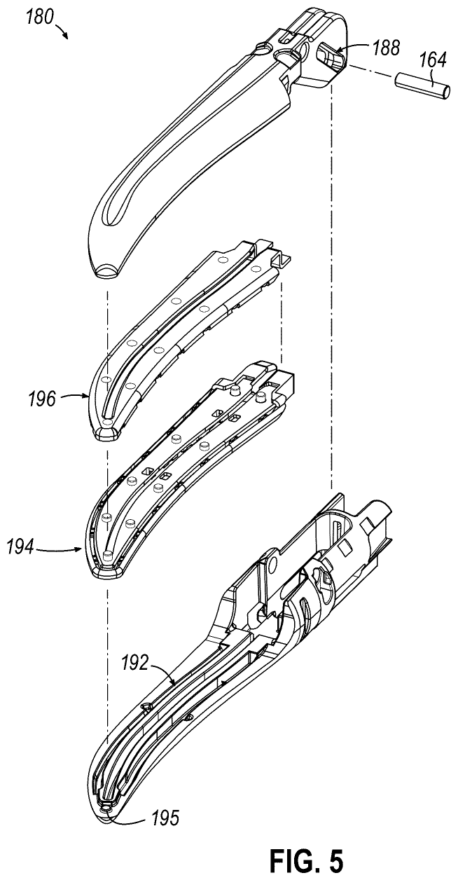

FIGS. 1-5 show a first example of an electrosurgical instrument (100). As best seen in FIG. 1, electrosurgical instrument (100) includes a handle assembly (120), a shaft assembly (140) having an articulation assembly (110), and an end effector (180). As will be described in greater detail below, end effector (180) of electrosurgical instrument (100) is operable to grasp, cut, and seal or weld tissue (e.g., a blood vessel, etc.). In the present example, end effector (180) is operable to seal or weld tissue by applying bipolar radiofrequency (RF) energy to the tissue. In some versions, end effector (180) is also configured to apply a non-therapeutic bipolar RF energy to identify and/or verify that the correct tissue is present in the end effector before a therapeutic RF energy can be applied to seal or weld tissue. In other versions, instrument (100) may alternatively be configured to seal or weld tissue via an ultrasonic blade, via staples, or in any other suitable fashion as would be apparent to one skilled in the art in view of the teachings herein.

In the present example, electrosurgical instrument (100) is electrically coupled to a waveform generator (200), which provides therapeutic and non-therapeutic energy, via power cable (10). Waveform generator (200) may be operable via a processor (265), which is incorporated into a monitor (202) having a graphical user interface (GUI) (203) in the present example. Waveform generator (200) may be configured to provide all or some of the electrical power requirements for use of electrosurgical instrument (100). By way of non-limiting example, the waveform generator (200) may be constructed and operable in accordance with at least some of the teachings of U.S. Pat. No. 8,986,302, entitled “Surgical Generator for Ultrasonic and Electrosurgical Devices,” issued Mar. 24, 2015, the disclosure of which is incorporated by reference herein, in its entirety. While electrosurgical instrument (100) is coupled to waveform generator (200) via power cable (10) in the present example, electrosurgical instrument (100) may alternatively contain one or more internal power sources (e.g., batteries and/or supercapacitors, etc.) to electrically power electrosurgical instrument (100); or may be powered in any other suitable fashion.

Handle assembly (120) is configured to be grasped by an operator with one hand, such that an operator may control and manipulate electrosurgical instrument (100) with a single hand. Although electrosurgical instrument (100) is primarily described herein as being used by a human user, alternative versions exist in which one or more robotic systems (e.g., a robotic arm) may be used to control and manipulate electrosurgical instrument (100). Shaft assembly (140) extends distally from handle assembly (120) and connects to articulation assembly (110). Articulation assembly (110) is also connected to a proximal end of end effector (180). As will be described in greater detail below, components of handle assembly (120) are configured to control end effector (180) such that an operator may grasp, cut, and seal or weld tissue. Articulation assembly (110) is configured to deflect end effector (180) laterally from the longitudinal axis (LA) defined by shaft assembly (140).

Handle assembly (120) of the present example includes a control unit (102) housed within a body (122), a pistol grip (124), a jaw closure trigger (126), a knife trigger (128), an activation button (130), an articulation control (132), and a knob (134). As will be described in greater detail below, jaw closure trigger (126) may be pivoted toward and away from pistol grip (124) to open and close jaws (182, 184) of end effector (180) to grasp tissue. Knife trigger (128) may be moved toward and away from pistol grip (124) and/or body (122) to actuate a knife member (176) within the confines of jaws (182, 184) to cut tissue captured between jaws (182, 184). Activation button (130) may be pressed to apply RF energy to tissue via electrodes (194, 196) of jaws (182, 184), respectively. In some versions, electrodes (194, 196) of jaws (182, 184) are in a bifurcation configuration where electrodes (194, 196) move relative to a central axis and nearly equal and opposite to one another.

Body (122) of handle assembly (120) defines an opening (123) through which a portion of articulation control (132) protrudes. Articulation control (132) is rotatably disposed within body (122) such that an operator may rotate the portion of articulation control (132) protruding from opening (123). Rotation of articulation control (132) relative to body (122) will bend articulation assembly (110) to thereby drive deflection of end effector (180) from the longitudinal axis (LA) defined by shaft assembly (140). Articulation control (132) and articulation assembly (110) may include any suitable features to drive deflection of end effector (180) from the longitudinal axis (LA) defined by shaft assembly (140) as would be apparent to one skilled in the art in view of the teachings herein.

Knob (134) is rotatably disposed on the distal end of body (122) and is configured to rotate end effector (180), articulation assembly (110), and shaft assembly (140) about the longitudinal axis (LA) of shaft assembly (140) relative to handle assembly (120). While in the current example, end effector (180), articulation assembly (110), and shaft assembly (140) are rotated by knob (134), knob (134) may be configured to rotate end effector (180) and articulation assembly (110) relative to selected portions of shaft assembly (140). Knob (134) may include any suitable features to rotate end effector (180), articulation assembly (110), and shaft assembly (140) as would be apparent to one skilled in the art in view of the teachings herein.

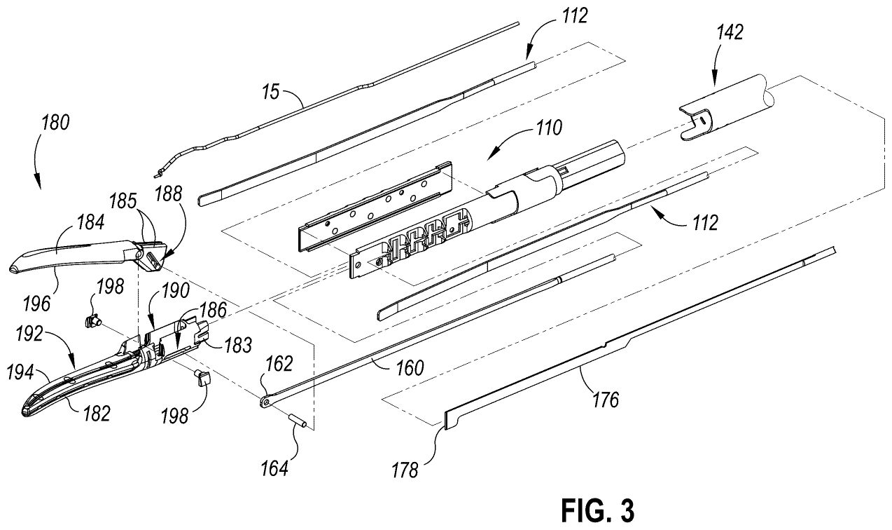

Shaft assembly (140) includes distal portion (142) extending distally from handle assembly (120) and a proximal portion housed within the confines of body (122) of handle assembly (120). As shown in FIG. 3, shaft assembly (140) houses a jaw closure connector (160) that couples jaw closure trigger (126) with end effector (180). Additionally, shaft assembly (140) houses a portion of knife member (176) extending between a distal cutting edge (178) of knife member (176) and knife trigger (128). Shaft assembly (140) also houses actuating members (112) that couple articulation assembly (110) with articulation control (132); as well as an electrical coupling (15) that operatively couples electrodes (194, 196) with activation button (130). As will be described in greater detail below, jaw closure connector (160) is configured to translate relative to shaft assembly (140) to open and close jaws (182, 184) of end effector (180); while knife member (176) is coupled to knife trigger (128) of handle assembly (120) to translate distal cutting edge (178) within the confines of end effector (180).

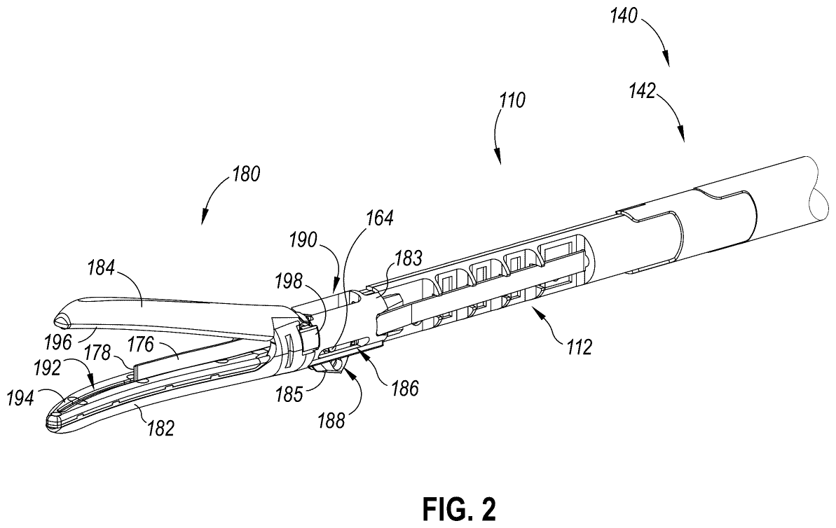

As best seen in FIGS. 2-5, end effector (180) includes lower jaw (182) pivotally coupled with upper jaw (184) via pivot couplings (198). Lower jaw (182) includes a proximal body (183) defining a slot (186), while upper jaw (184) includes proximal arms (185) defining a slot (188). Lower jaw (182) also defines a central channel (190) that is configured to receive proximal arms (185) of upper jaw (184), portions of knife member (176), jaw closure connector (160), and pin (164). Slots (186, 188) each slidably receive pin (164), which is attached to a distal coupling portion (162) of jaw closure connector (160).

Lower jaw (182) includes a force sensor (195) located at a distal tip of lower jaw (182), though force sensor (195) may alternatively be positioned at any other suitable location. Force sensor (195) may be in communication with control unit (102). Force sensor (195) may be configured to measure the closure force generated by pivoting jaws (182, 184) into a closed configuration in accordance with the description herein. Force sensor (195) may communicate this data to control unit (102). Any suitable components may be used for force sensor (195) as would be apparent to one skilled in art in view of the teachings herein. In some variations, end effector (180) includes more than one force sensor; while in other variations force sensor (195) is omitted.

Jaw closure connector (160) is operable to translate within central channel (190) of lower jaw (182). Translation of jaw closure connector (160) drives pin (164). With pin (164) being located within both slots (186, 188), and with slots (186, 188) being angled relative to each other, pin (164) cams against proximal arms (185) to pivot upper jaw (184) toward and away from lower jaw (182) about pivot couplings (198). Therefore, upper jaw (184) is configured to pivot toward and away from lower jaw (182) about pivot couplings (198) to grasp tissue.

Lower jaw (182) and upper jaw (184) also define a knife pathway (192). Knife pathway (192) is configured to slidably receive knife member (176), such that knife member (176) may be retracted, and advanced, to cut tissue captured between jaws (182, 184).

Lower jaw (182) and upper jaw (184) each comprise a respective electrode (194, 196). Waveform generator (200) may provide RF energy to electrodes (194, 196) via electrical coupling (15) that extends through handle assembly (120), shaft assembly (140), articulation assembly (110); and that electrically couples with one or both of electrodes (194, 196). Waveform generator (200) may selectively activate electrodes (194, 196) via electrical coupling (15) in response to an operator pressing activation button (130). In some instances, control unit (102) may couple electrical coupling (15) with activation button (130), such that control unit (102) activates electrodes (194, 196) in response to operator pressing activation button (130). Control unit (102) may have any suitable components i to perform suitable functions as would be apparent to one skilled in the art in view of the teachings herein. For instance, control unit (102) may have a processor, memory unit, suitable circuitry, etc. Examples of features and functionalities that may be incorporated into control unit (102) will be described in greater detail below. Control unit (102) and processor (265) may include overlapping and interchangeable functions such that either may be capable of performing functions of the other described herein.

As described above, jaw closure trigger (126) may be pivoted toward and away from pistol grip (124) to open and close jaws (182, 184) of end effector (180) to grasp tissue. Pivoting jaw closure trigger (126) toward pistol grip (124) may proximally actuate jaw closure connector (160) and pin (164), which in turn cams against slots (188) of proximal arms (185) of upper jaw (184), thereby rotating upper jaw (184) about pivot couplings (198) toward lower jaw (182) such that jaws (182, 184) achieve a closed configuration.

In some versions, knife trigger (128) may be moved toward and away from body (122) and/or pistol grip (124) to actuate knife member (176) within knife pathway (192) of jaws (182, 184) to cut tissue captured between jaws (182, 184). To that end, handle assembly (120) includes a knife coupling body (not shown) that is slidably coupled along proximal portion of shaft assembly (140). The knife coupling body is coupled with knife member (176) such that translation of knife coupling body relative to proximal portion of shaft assembly (140) translates knife member (176) relative to shaft assembly (140). Knife member (176) includes distal cutting edge (178) that is configured to sever tissue captured between jaws (182, 184). Therefore, actuating knife trigger (128) causes knife member (176) to actuate within knife pathway (192) of end effector (180) to sever tissue captured between jaws (182, 184).

With distal cutting edge (178) of knife member (176) actuated to the advanced position, an operator may press activation button (130) to selectively activate electrodes (194, 196) of jaws (182, 184) to seal or weld severed tissue captured between jaws (182, 184). The operator may also press activation button (130) to selectively activate electrodes (194, 196) of jaws (182, 184) at any suitable time during use (e.g., while knife member (176) is in a proximal position). The operator may release jaw closure trigger (126), such that jaws (182, 184) pivot into the opened configuration, releasing tissue.

### II. Sensing Tissue Impedance for Determinations of Tissue State

Electrosurgical instrument (100) discussed above is configured to clamp tissue within end effector (180). With the tissue securely clamped in end effector (180), electrodes (194, 196) may apply non-therapeutic energy in the form of a low voltage waveform to the tissue; and sensor devices may measure the return signal (i.e., the waveform) to calculate and measure the impedance and reactance of the tissue. In some versions, electrodes (194, 196) are operatively connected to such sensor devices such that electrodes (194, 196) may be referred to as sensors in this respect. More specifically, electrosurgical instrument (100), via one or more sub-circuits, will provide non-therapeutic energy to the extracellular and intracellular fluid present within a given (e.g., clamped) region of tissue to determine an impedance of the tissue within jaws (182, 184). A processor of waveform generator (200) and/or a processor of control unit (102) may then relay information associated with the impedance and reactance. Using this associated information, a system inclusive of such electrosurgical instrument (100) may identify the impedance and reactance of the tissue clamped between jaws (182, 184); and determine the optimal signal characteristics for the therapeutic signal and/or if a proper seal has been created after applying the therapeutic RF energy. In some such cases, processor (265) may determine when tissue has been sufficiently clamped; and based on this determination, determine whether to provide therapeutic energy.

### III. Second Example of an Electrosurgical Instrument

In some situations, it may be beneficial to provide a version of electrosurgical instrument (100) that is operable in different selectable modes of operation. For instance, a first mode of operation may be provided where the operator actuates jaw closure trigger (126) first to clamp tissue in end effector (180); then actuates activation button (130) while holding jaw closure trigger (126) in an actuated configuration to apply RF energy to tissue clamped by end effector (180). A second mode of operation may be provided where the operator drives jaw closure trigger (126) through a first range of motion to clamp tissue in end effector (180); then drives jaw closure trigger (126) through a second range of motion to apply RF energy to tissue clamped by end effector (180). Thus, in the second mode of operation, the operator may not need to actuate activation button (130) to apply RF energy to tissue clamped by end effector (180). A third mode of operation may be provided where the operator actuates jaw closure trigger (126) first to clamp tissue in end effector (180); the jaw closure trigger (126) is latched to maintain the actuated position; then the operator actuates activation button (130) to apply RF energy to tissue clamped by end effector (180). Thus, in the third mode of operation, the operator may not need to continue holding jaw closure trigger (126) in the actuated position to maintain the actuated position.

A mode of operation may be selected according to a particular user preference or may be beneficial for a particular treatment type. Offering different modes of operation for the same electrosurgical instrument (100) may also be beneficial to reduce the total number of instruments used for a particular surgical operation as multiple instruments, each having a unique mode of operation, may no longer be needed. An example of a multi-mode version of electrosurgical instrument (100) is described in greater detail below.

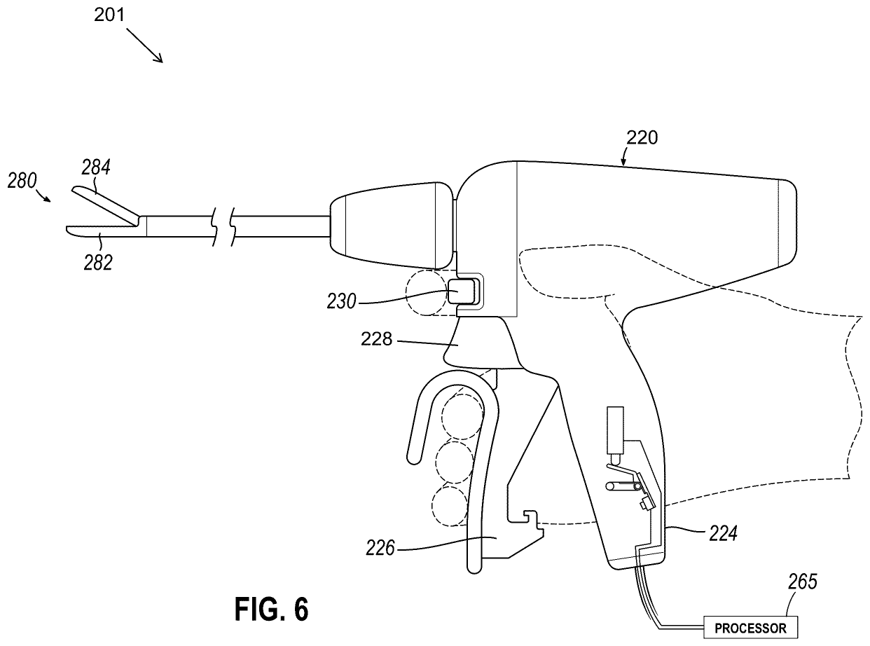

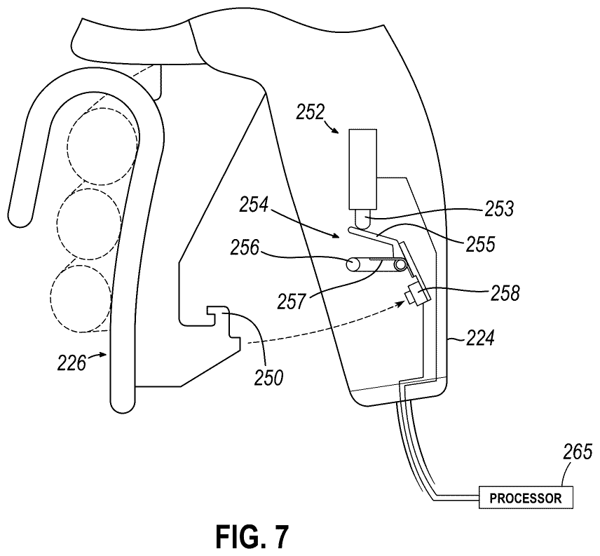

FIGS. 6-7 illustrate a second example of an electrosurgical instrument (201) which may be substantially similar in form and function to electrosurgical instrument (100) except for the differences described below. Electrosurgical instrument (201) includes a handle assembly (220) having a pistol grip (224), a jaw closure trigger (226), a knife trigger (228) and an activation button (230). A shaft assembly (240) extends distally from handle assembly (220) and has an end effector (280) at a distal end. End effector (280) has a lower jaw (282) and an upper jaw (284). These components may be constructed and operable like the similarly named components of electrosurgical instrument (100) in that, as trigger (226) is pivoted toward pistol grip (224), upper jaw (284) pivots toward lower jaw (282) to thereby grasp tissue to be sealed using activation button (230). In cases where the operator wishes to sever the tissue captured in end effector (280), the operator may actuate knife trigger (228).

As shown in FIG. 7, internal components of pistol grip (224) include a solenoid (252) (also referred to as an actuator), a lock (254), a switch (258), and a trigger latch (250) of trigger (226). Here, solenoid (252) is in electrical communication with processor (265) of monitor (202) and is thus operable to actuate a rod (253) between a retracted position and an extended position. While solenoid (252) is shown including rod (253), any other type of solenoid or actuator may be used. By way of example only, an electromagnet, motorized assembly, and/or other electrically actuated locking feature may be used. In the present example, when solenoid (252) provides rod (253) in the retracted position, this arrangement allows lock (254) to transition into an unlocked configuration. When solenoid (252) drives rod (253) to the extended position, this arrangement drives lock (254) to transition into a locked configuration. While it is envisioned that this functionality could be reversed where the retracted position of rod (253) results in lock (254) being in the unlocked configuration and where the extended position of rod (253) results in lock (254) being in the locked configuration, in either arrangement solenoid (252) may include a failsafe position that results in lock (254) being in the unlocked configuration. In other words, should solenoid (252) experience a loss of power, lock (254) may transition or remain in the unlocked configuration.

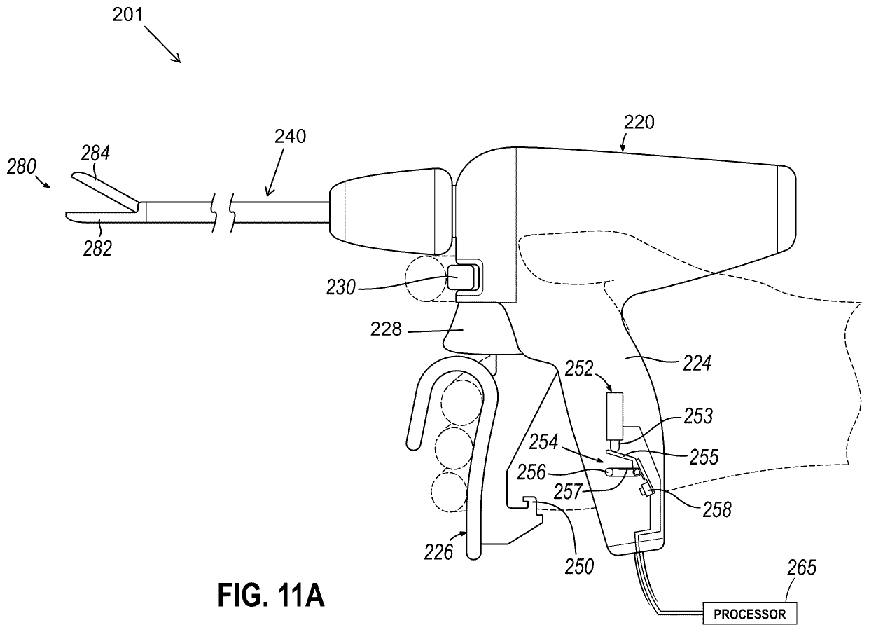

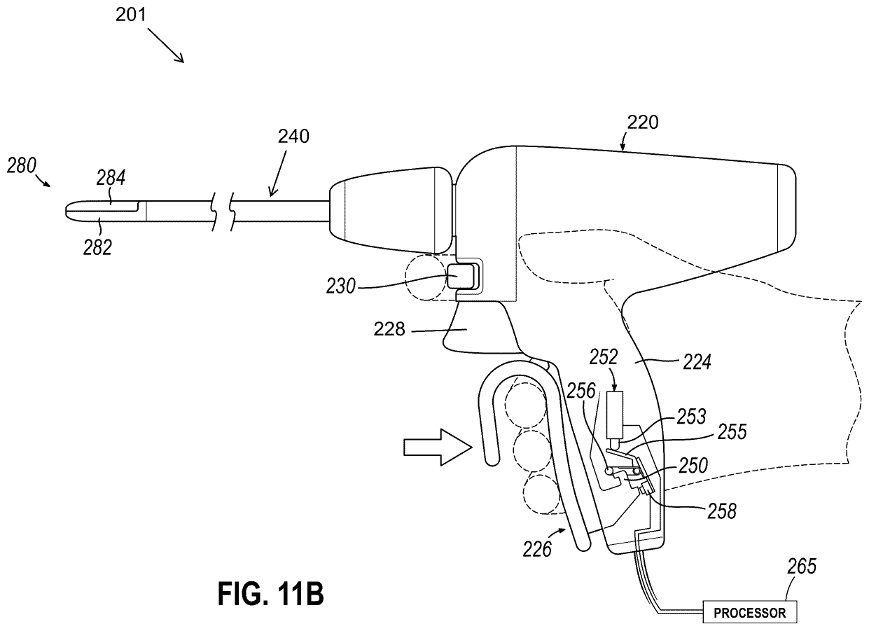

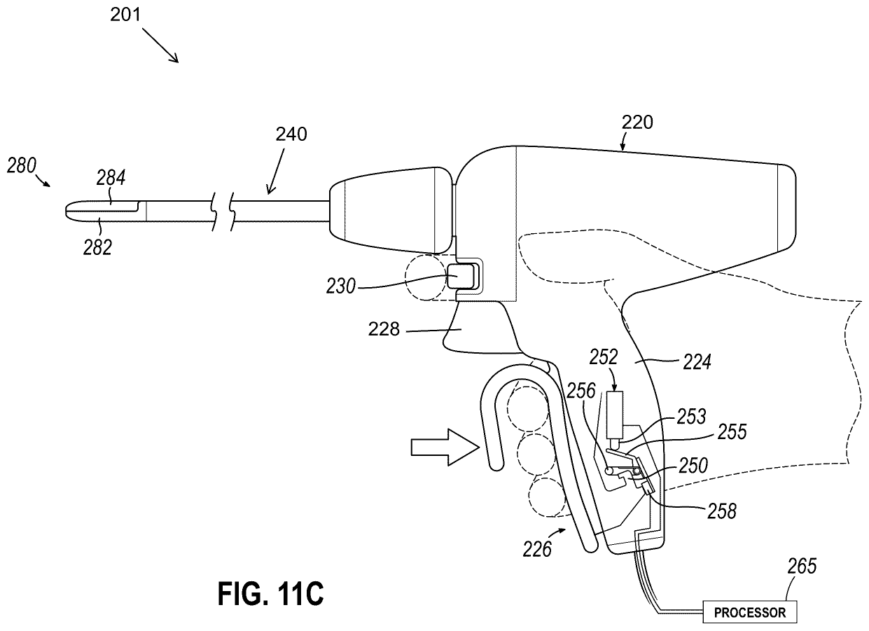

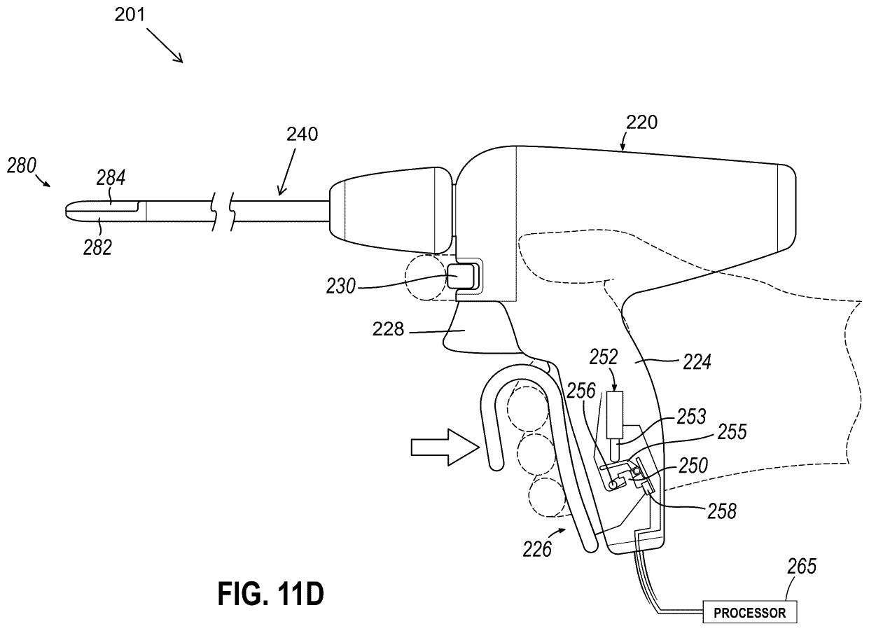

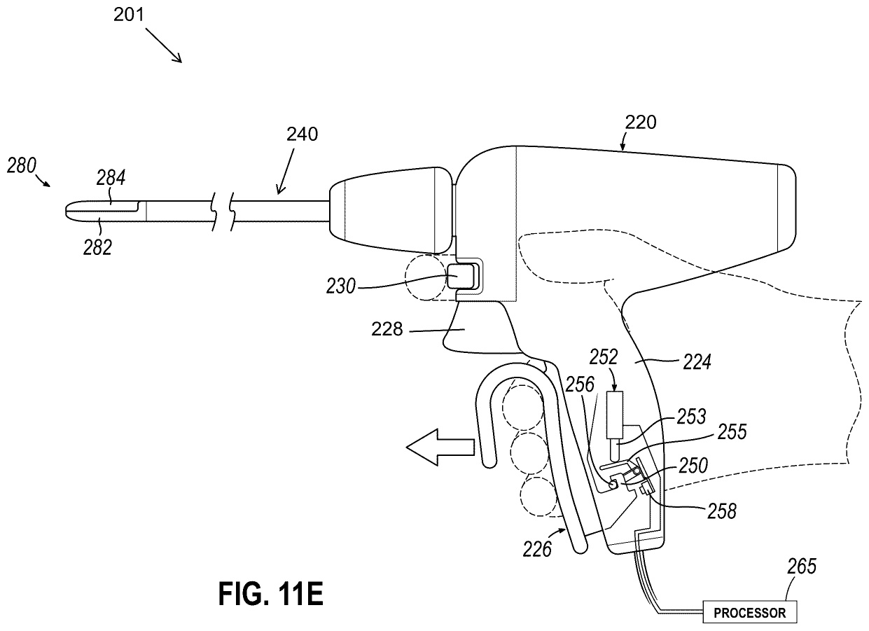

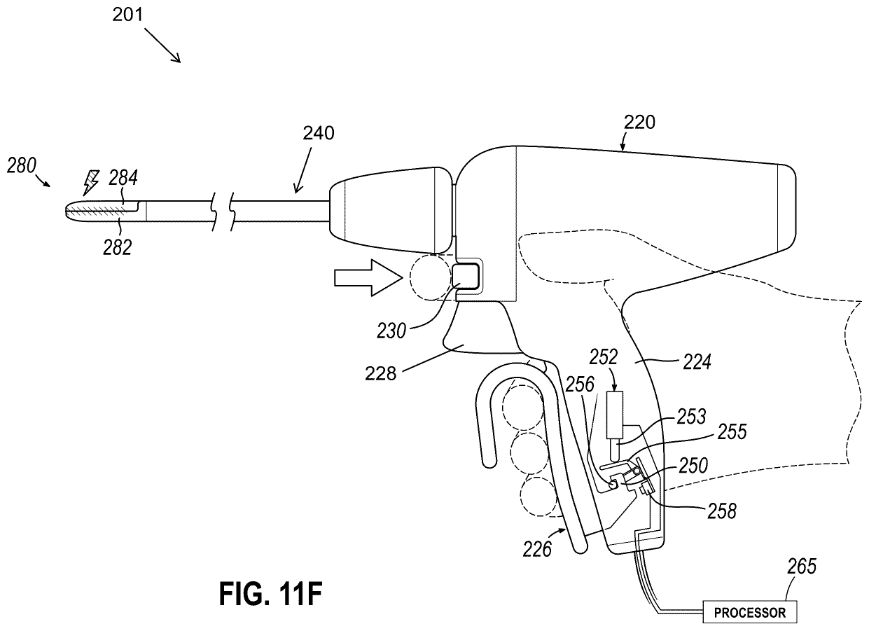

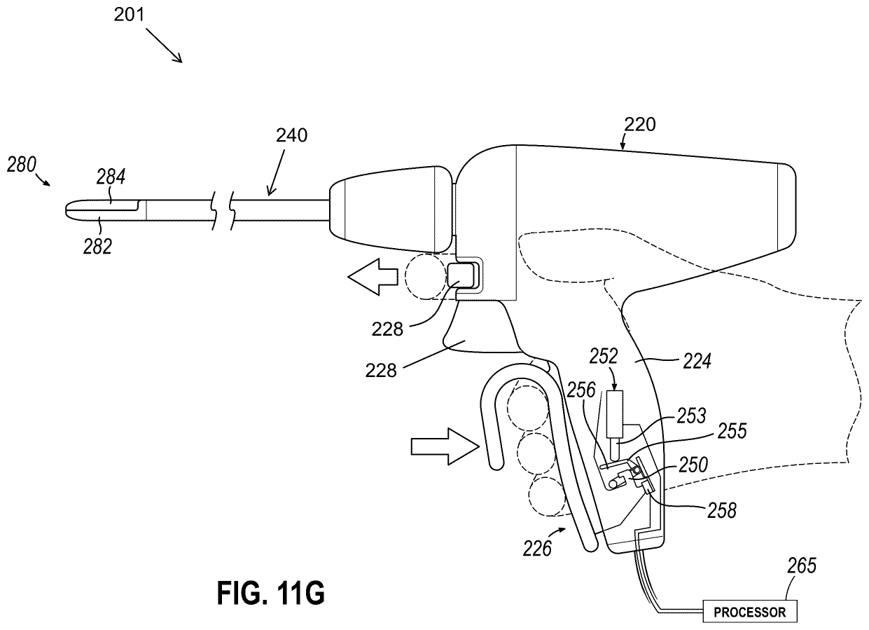

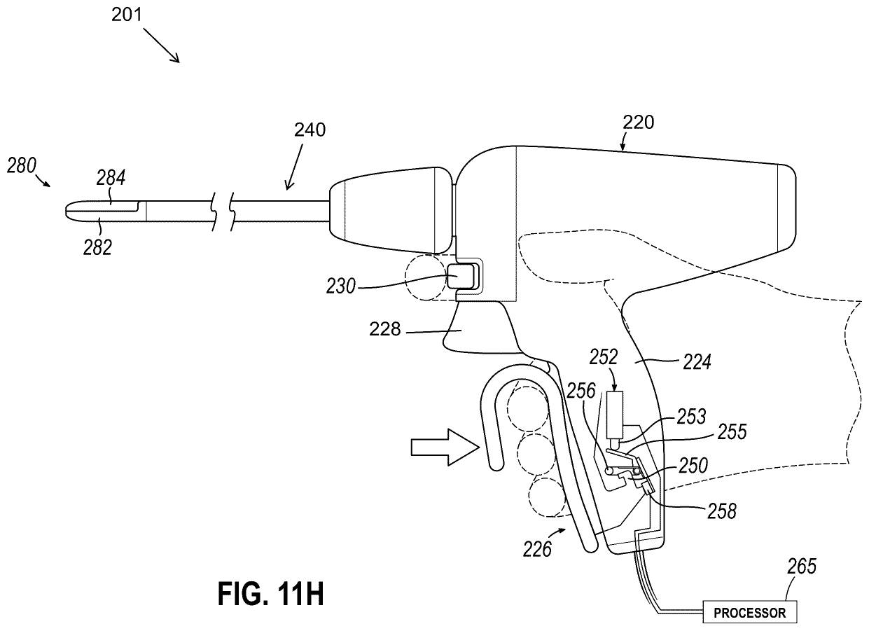

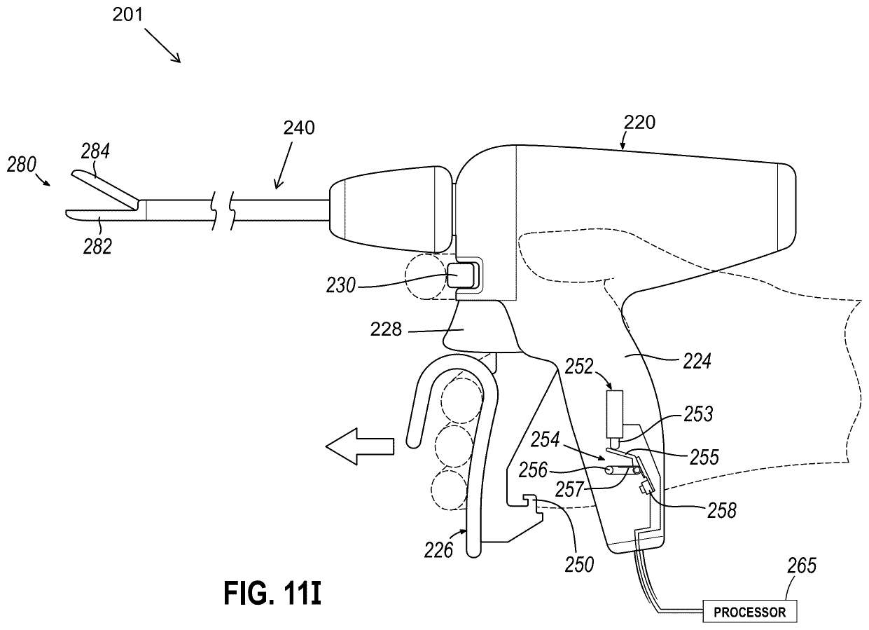

Lock (254) includes a lock arm (255), a lock pin (256), and a lock spring (257). Lock (254) is shown as being pivotable relative to the pistol grip (224) between the locked configuration (FIGS. 11D-11G) and the unlocked configuration (FIGS. 11A-11C and 11H-11I). Lock spring (257), shown as a torsion spring, is sized and positioned to bias lock (254) into the unlocked configuration upon solenoid rod (253) return to the retracted position. Solenoid (252) and lock spring (257) are also sized accordingly such that solenoid (252) may overcome the biasing force from lock spring (257) to thereby transition lock (254) into the locked configuration upon a command from processor (265). Alternatively, lock spring (257) may be incorporated into solenoid (252) to thereby bias solenoid rod (253) into the retracted position and solenoid rod (253) may also be affixed to lock arm (255) such that lock (254) is biased into the unlocked configuration upon a loss of power to solenoid (252). Any other suitable biasing features may be used, such that lock spring (257) may be omitted in some versions.

Lock pin (256) thus transitions between the locked and unlocked configurations based on the resulting force from solenoid (252) and lock spring (257). When in the locked configuration (see FIGS. 11D-11G) lock pin (256) is positioned to engage with a trigger latch (250) of trigger (226). Lock pin (256) is shown as comprising a laterally directed, cantilevered pin sized to fit within trigger latch (250). As shown, trigger latch (250) is hook-shaped such that, when engaged with lock pin (256) (FIGS. 11E-11F) trigger (226) is prevented from returning to a released position that is away from pistol grip (224). As described above, a loss of power to processor (265)/solenoid (252) could result in lock (254) biasing towards the unlocked position. Once lock pin (256) is hooked by and engages with trigger latch (250), the biased transition towards the unlocked configuration is prevented since lock pin (256) is prevented from moving upwards by trigger latch (250) (as shown). In such a situation, trigger latch (250) may be released from lock pin (256) by pulling trigger (226) proximally toward pistol grip (224), allowing lock (254) to resiliently return into the unlocked configuration under the influence of lock spring (257), with lock pin (256) out of a travel path of trigger latch (250). Trigger (226) may then be released to thus return to the released position. Such a design may be beneficial to maintain clamped tissue in end effector (280) until power can be restored while also allowing for a selective opening of end effector (280). In other words, end effector (280) will not suddenly and unexpectedly transition from a closed configuration to an open configuration in the event of a power loss.

Switch (258) is in electrical communication with processor (265) such that processor (265) is capable of sensing when switch (258) has been pressed. Switch (258) is positioned along pistol grip (224) such that it is within a travel path of trigger (226). Specifically, switch (258) is pressed by trigger (226) when trigger (226) has been pulled to the most proximal position (FIG. 11C). Once trigger (226) has pressed switch (258), processor (265) then senses that switch (258) has been pressed and is thus able to determine the position of trigger (226) relative to pistol grip (224). Switch (258) may provide tactile and/or audible feedback such that a user can feel and/or hear when trigger (226) has pressed switch (258). Further, processor (265) may be configured to generate an audible response and/or to indicate that switch (258) has been pressed on monitor (202). Such audible and/or tactile feedback indicating depression of switch (258) may be provided in addition to the above-described audible and/or tactile feedback indicating that trigger (226) has reached the position shown in FIG. 11B; before reaching the position shown in FIG. 11C.

Switch (258) may also be normally open or normally closed, based on the configuration of processor (265). While switch (258) is shown as a momentary switch, other types of switches are envisioned that would be capable of detecting a position of trigger (226). Specifically, while switch (258) as shown is activated by trigger (226) reaching a specific proximal-most position, other switches may be capable of determine trigger (226) position throughout an entire range of movement, such as through use of a potentiometer, rotary switch, encoder, etc. In such an example, processor (265) would still be capable of determining when trigger (226) is in the proximal-most position.

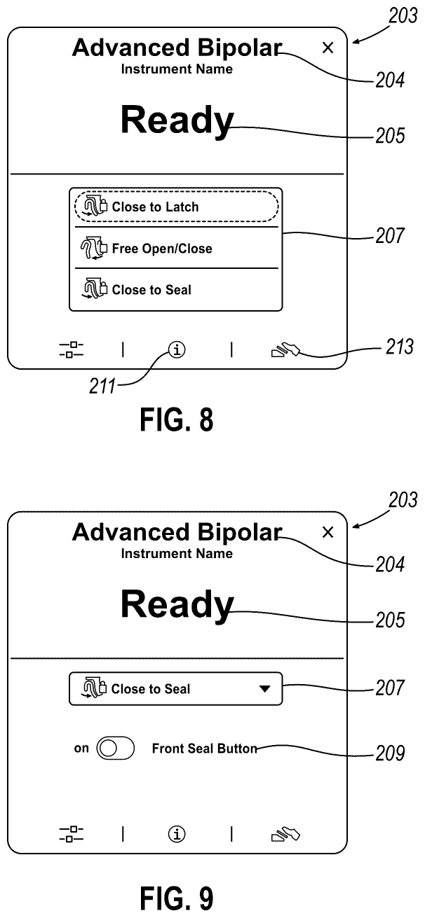

FIGS. 8-9 show various pages of GUI (203) that may be used to select and communicate various modes in which processor (265) may operate. Operation of each mode will be described in detail below. GUI (203) includes instrument indicator (204), status indicator (205), mode selector (207), information selector (211), and settings selector (213).

Instrument indicator (204) is configured to indicate to a user which instrument is communicating with processor (265). In the present example, an advanced bipolar instrument in the form of electrosurgical instrument (201) is in communication with processor (265).

Status indicator (205) is configured to indicate a status of electrosurgical instrument (201), such as whether the instrument is ready for use or not. Specific indications may include that the instrument is: ready for use, processing, faulty, unknown, or if a selection is needed in order to operate. Any other status apparent to a person skilled in the art in view of the teachings herein may also be indicated by status indicator (205).

Mode selector (207) is shown as a dropdown menu that allows a user to select a particular mode by which processor (265) is to operate. As shown, mode selector (207) for electrosurgical instrument (201) may include “Close to Latch,” “Free Open/Close,” and “Close to Seal” modes. Operation of each of these listed modes will be described in detail below.

Information selector (211) is configured to allow a user to view information specific to electrosurgical instrument (201) and processor (265), such as hardware and software versions, etc.

Settings selector (213) is configured to allow a user to toggle certain settings of processor (265) that can thereby change settings of electrosurgical instrument (201), processor (265), waveform generator (200), monitor (202), GUI (203), and any other peripheral devices in communication with processor (265). Processor (265) includes a memory (not shown) capable of storing specific preferences for each user. Stored preferences may include the last and preferred mode of operation for the particular user.

FIG. 9 shows a rendering by GUI (203) indicating the selected mode and any specific options associated with the selected mode. As a specific example and as shown, the “Close to Seal” mode has been selected and an additional Front Seal Button toggle button has been presented to the user. The Front Seal Button toggle button may be specific to the “Close to Seal” mode.

GUI (203) thus allows the user to select between various modes from which processor (265) may control electrosurgical instrument (201). Each mode may function differently from any other mode and may utilize different aspects of electrosurgical instrument (201). Described below are examples of details for each of the above listed modes. One commonality between each or any of the modes may be that processor (265) allows for therapeutic energy to be delivered via electrodes (194, 196) once tissue has been sensed as described above. Processor (265) may also be capable of overriding an RF activation command from a user if tissue is not sensed between electrodes (194, 196).

### A. Close to Latch Mode

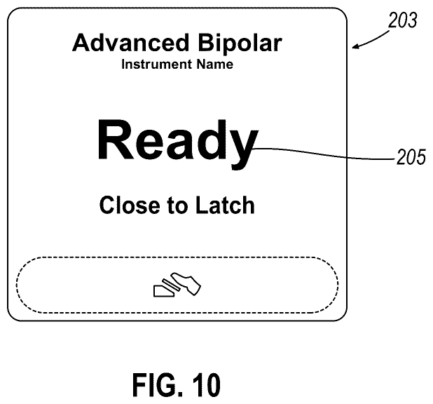

FIG. 10 illustrates what GUI (203) may show to a user upon a selection of the “Close to Latch” mode. Once status indicator (205) instructs a user that electrosurgical instrument (201) is ready for use, a user may begin operation.

FIGS. 11A-11I show an example of operation of electrosurgical instrument (201) when processor (265) is in “Close to Latch” mode. In the state shown in FIG. 11A, jaws (282, 284) of end effector (280) are in an open configuration such that tissue may enter between jaws (282, 284). Trigger (226) is in a fully distal position (released position). Solenoid (252) is in a non-activated state, such that rod (253) is in the retracted position, and such that lock (254) is in the unlocked configuration. Switch (258) is unpressed.

In the state shown in FIG. 11B, trigger (226) has been actuated or retracted from the released position to an actuated position such that end effector (280) is in the closed configuration. However, switch (258) has still not yet been pressed in the state shown in FIG. 11B. Thus, end effector (280) may grasp tissue to some degree before switch (258) is pressed.

In the state shown in FIG. 11C, trigger (226) has been further actuated or retracted to a proximal-most position such that trigger (226) has pressed switch (258). With switch (258) now pressed, processor (265) determines that trigger (226) is in the proximal-most position. In some cases, in the transition between the state shown in FIG. 11B and the state shown in FIG. 11C, end effector (280) applies an increasing clamping force on the tissue captured between jaws (282, 284). In some other versions, one or more deforming features, slip features, or other features deform or otherwise move in response to trigger (226) transitioning from the position shown in FIG. 11B to the position shown in FIG. 11C without the clamping force on the tissue being increased. Some versions of electrosurgical instrument (201) may include a detent feature and/or other feature that provides audible and/or tactile feedback to indicate when trigger (226) has reached the position shown in FIG. 11B, before trigger (226) is further actuated to reach the position shown in FIG. 11C. This may facilitate the user in arresting motion of trigger (226), if desired, after achieving some degree of closure of jaws (282, 284) but before switch (258) is pressed.

In the state shown in FIG. 11D, processor (265) has activated solenoid (252) in response to the pressing of switch (258), such that solenoid (252) has driven rod (253) to the extended position to thereby drive lock (254) into the locked configuration. Lock pin (256) has moved downwards to thereby be distal to trigger latch (250). In some versions, the transition from the state shown in FIG. 11C and the state shown in FIG. 11D is substantially instantaneous, with processor (265) immediately activating solenoid (252) in response to the pressing of switch (258). In some other versions, one or more other conditions are required to cause processor (265) to activate solenoid (252) after switch (258) has been pressed. By way of example only, one or more sensors in end effector (280) may sense the presence of tissue between jaws (282, 284), and processor (265) may further require a signal from such a sensor indicating that tissue is captured between jaws (282, 284) for processor (265) to activate solenoid (252) in response to the pressing of switch (258). Alternatively, any other suitable conditions may be required for processor (265) to activate solenoid (252) when switch (258) has been pressed.

In the state shown in FIG. 11E, the user has at least partially released their grasp on trigger (226), such that trigger (226) has moved distally, and such that trigger latch (250) has now engaged with lock pin (256). In this orientation, the user is free to remove their grip from trigger (226) and still maintain a grip on tissue with end effector (280).

In the state shown in FIG. 11F, the user has pressed activation button (230) to thereby energize electrodes of jaws (282, 284) (e.g., similar to electrodes (194, 196)) and thereby seal tissue. As described above, processor (265) may be capable of ceasing or preventing energizing of the electrodes, despite the user pressing activation button (230), if no tissue is sensed between jaws (282, 284). While not shown, in some uses of electrosurgical instrument (201), the user may actuate knife trigger (228) to sever tissue captured between jaws (282, 284) before pressing activation button (230) to seal the tissue with bipolar RF energy. While activation button (230) is provided in the present example, some variations may include a footswitch and/or another external activation source that may be used to energize electrodes of jaws (282, 284), in addition to or in lieu of providing activation button (230).

In the state shown in FIG. 11G, the user has released activation button (230) to thereby stop the energizing of the electrodes of jaws (282, 284). The user has also further pulled or otherwise actuated trigger (226) further proximally, to thereby press switch (258) again. Releasing activation button (230) and pulling trigger (226) further proximally as shown in FIG. 11G may be performed independently of each other. Processor (265) may be configured to cease RF activation upon sensing that switch (258) has been pressed and/or in response to sensing that activation button (230) has been released. Processor (265) may also be configured to transition lock (254) to the unlocked configuration upon sensing that activation button (230) has been released and/or in response to sensing that switch (258) has been pressed.

In the state shown in FIG. 11H, lock (254) has transitioned into the unlocked configuration such solenoid (252) has been deactivated and rod (253) is in the retracted position. Lock pin (256) is resiliently urged by spring (257) away from the path of trigger latch (250). In some versions, the transition from the state shown in FIG. 11G and the state shown in FIG. 11H is substantially instantaneous, with processor (265) immediately deactivating solenoid (252) in response to the second pressing of switch (258) and/or the release of activation button (230). In some other versions, one or more other conditions are required to cause processor (265) to deactivate solenoid (252) after switch (258) has been pressed and/or activation button (230) has been released. By way of example only, one or more sensors in end effector (280) may sense the impedance of tissue between jaws (282, 284), and processor (265) may further require a signal from such a sensor indicating that the tissue has been sufficiently sealed (based on the impedance signal) for processor (265) to deactivate solenoid (252) in response to the pressing of switch (258) and/or the release of activation button (230). Alternatively, any other suitable conditions may be required for processor (265) to deactivate solenoid (252) when switch (258) has been pressed and/or activation button (230) has been released.

In the state shown in FIG. 11I, trigger (226) has been returned to the distal-most position such that end effector (280) is open and no longer grasps tissue. At this stage, the above process may be repeated at a different region of tissue. Alternatively, the user may select a different mode of operation for electrosurgical instrument (201) and use electrosurgical instrument (201) in the different selected mode. Alternatively, the process may be complete.

### B. Free Open/Close Mode

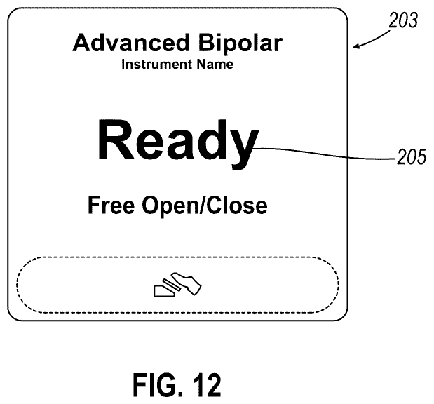

FIG. 12 illustrates what GUI (203) may show to a user upon a selection of the “Free Open/Close” mode. Once status indicator (205) instructs a user that electrosurgical instrument (201) is ready for use, the user may begin operation.

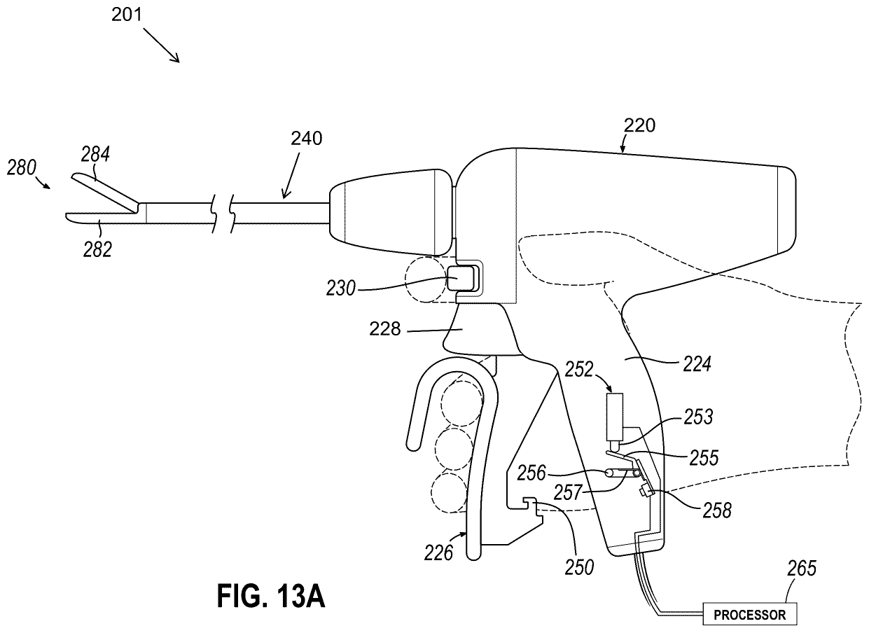

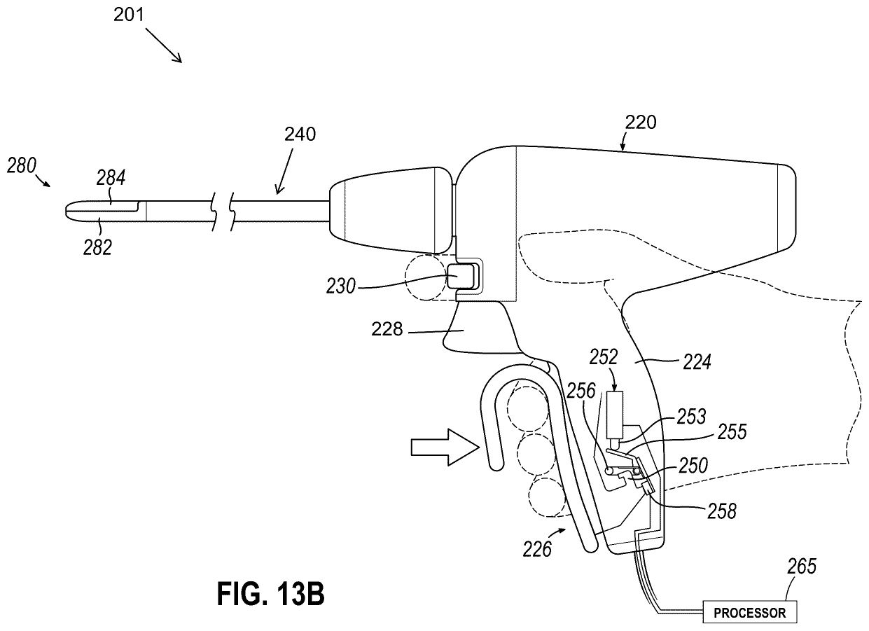

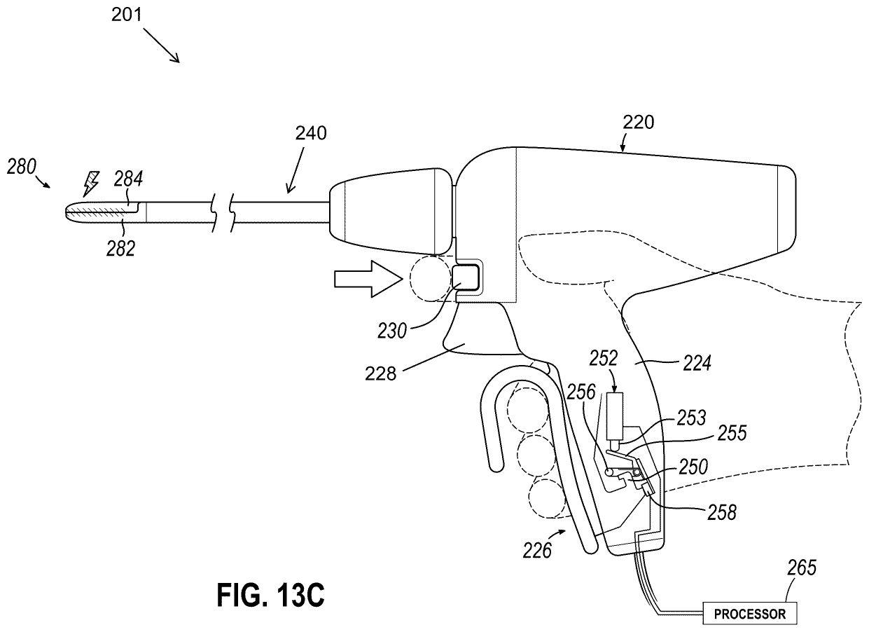

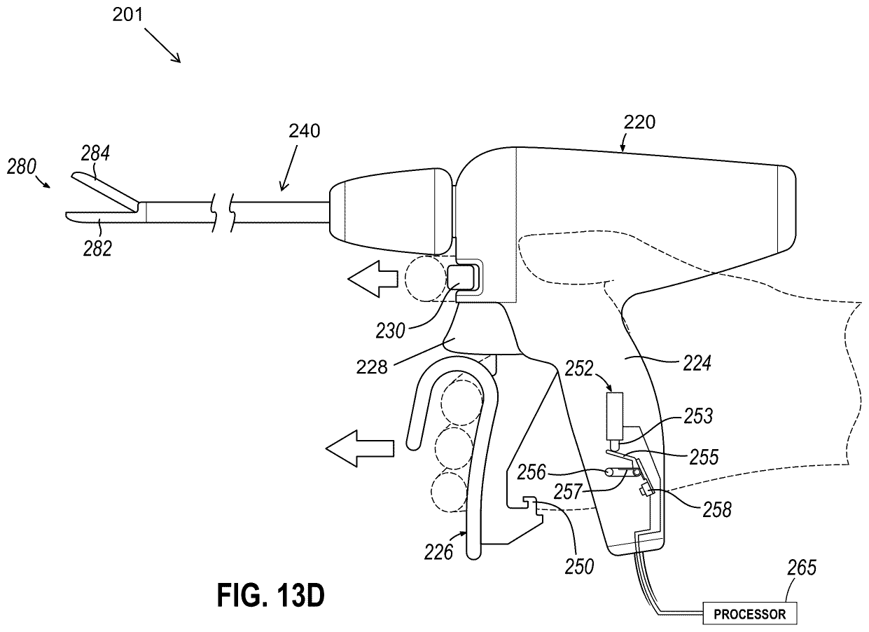

FIGS. 13A-13D show an example of operation of electrosurgical instrument (201) when processor (265) is in “Free Open/Close” mode. In the state shown in FIG. 13A, jaws (282, 284) of end effector (280) are in an open configuration such that tissue may enter between jaws (282, 284). Trigger (226) is in a fully distal position (released position). Solenoid (252) is in a non-activated state, such that rod (253) is in the retracted position, and such that lock (254) is in the unlocked configuration. Switch (258) is unpressed.

In the state shown in FIG. 13B, trigger (226) has been fully actuated or retracted from the released position to an actuated position such that end effector (280) is in the closed configuration and switch (258) is pressed thereby signaling processor (265) that trigger (226) is in the proximal-most position (a pressed position). Unlike the “Close to Latch” mode described above with respect to FIGS. 11A-11I, in the “Free Open/Close” mode of FIGS. 13A-13D, pressing of switch (258) does not cause activation of solenoid (252). Lock (254) thus remains in the unlocked configuration despite trigger (226) being fully actuated. While pressing of switch (258) does not cause activation of solenoid (252) in this example, pressing of switch (258) may have other effects in some versions. For instance, processor (265) may prevent activation of electrodes of jaws (282, 284) unless switch (258) is being depressed in some versions. Alternatively, pressing of switch (258) may have other effects; or may have no effect at all on operation of electrosurgical instrument (201) in some versions of the “Free Open/Close” mode.

In the state shown in FIG. 13C, the user has pressed activation button (230) to thereby energize electrodes of jaws (282, 284) and thereby seal tissue. As described above, processor (265) may be capable of ceasing or preventing energizing of the electrodes, despite the user pressing activation button (230), if no tissue is sensed between jaws (282, 284). While not shown, in some uses of electrosurgical instrument (201), the user may actuate knife trigger (228) to sever tissue captured between jaws (282, 284) before pressing activation button (230) to seal the tissue with bipolar RF energy.

In the state shown in FIG. 13D, the user has released activation button (230) to thereby stop the energizing of the electrodes of jaws (282, 284). The user has also released trigger (226) and to unclamp tissue held within end effector (280). Should activation button (230) remain pressed while trigger (226) moves distally and releases from switch (258), processor (265) may automatically end RF activation or may activate solenoid (252) to thereby transition lock (254) into the locked configuration, to thereby prevent a release of tissue from end effector (280) until RF activation has ended. Solenoid (252) and lock (254) may also be inoperable in this mode. In any of these scenarios, after the tissue has been released from end effector (280), the above process may be repeated at a different region of tissue. Alternatively, the user may select a different mode of operation for electrosurgical instrument (201) and use electrosurgical instrument (201) in the different selected mode. Alternatively, the process may be complete.

### C. Close to Seal Mode

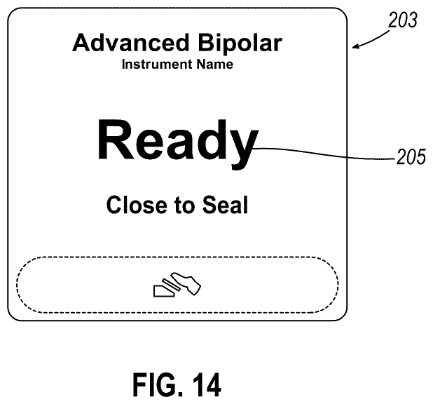

FIG. 14 illustrates what GUI (203) shows a user upon a selection of the “Close to Seal” mode. Once status indicator (205) instructs a user that electrosurgical instrument (201) is ready for use, a user may begin operation. As shown previously in FIG. 9, “Close to Seal” mode may optionally include Front Seal Button toggle button.

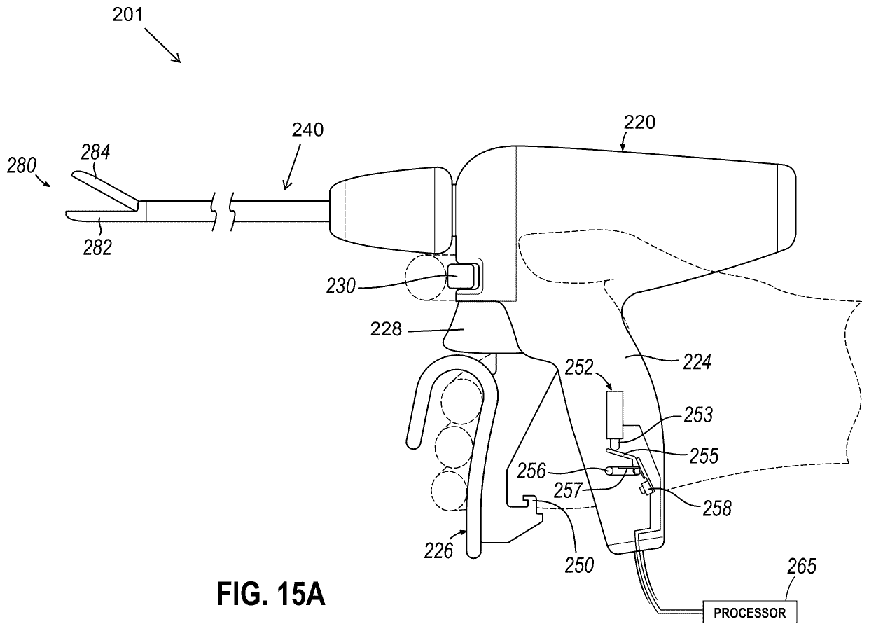

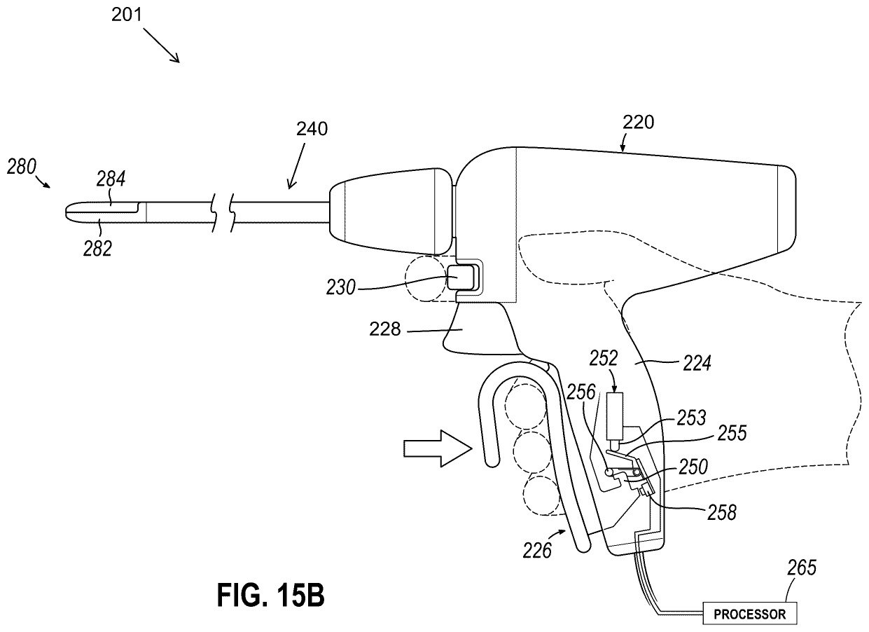

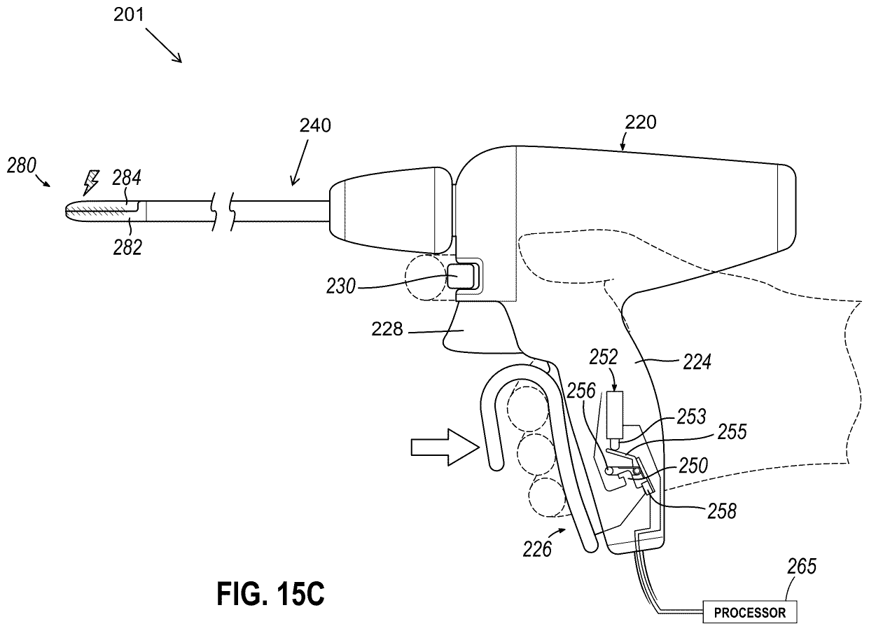

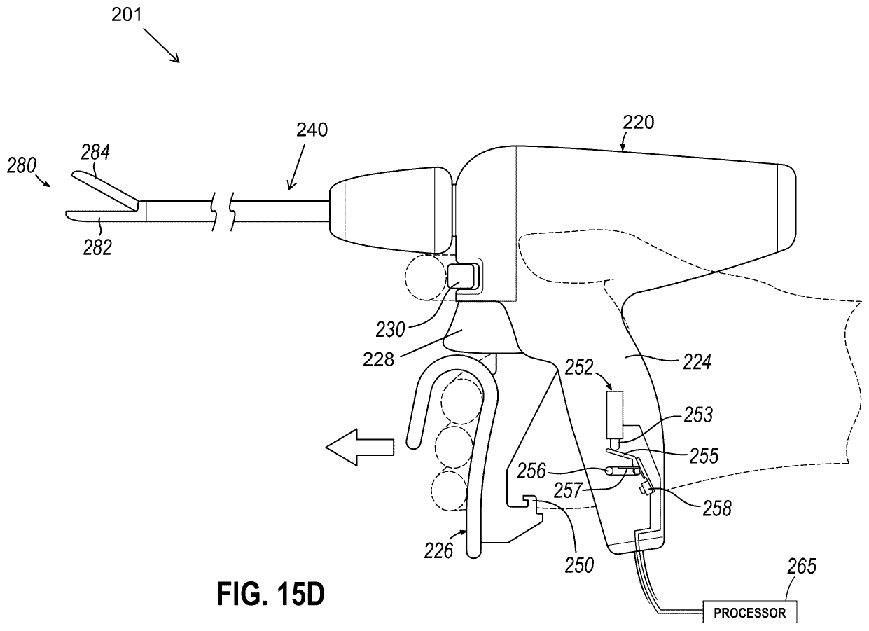

FIGS. 15A-15D show an example of operation of electrosurgical instrument (201) when processor (265) is in “Close to Seal” mode. In the state shown in FIG. 15A, jaws (282, 284) of end effector (280) are in an open configuration such that tissue may enter between jaws (282, 284). Trigger (226) is in a fully distal position (released position). Solenoid (252) is in a non-activated state, such that rod (253) is in the retracted position, and such that lock (254) is in the unlocked configuration. Switch (258) is unpressed.

In the state shown in FIG. 15B, trigger (226) has been actuated or retracted from the released position to an actuated position such that end effector (280) is in the closed configuration. However, switch (258) has still not yet been pressed in the state shown in FIG. 15B. Thus, end effector (280) may grasp tissue to some degree before switch (258) is pressed.

In the state shown in FIG. 15C, trigger (226) has been further actuated or retracted to a proximal-most position such that trigger (226) has pressed switch (258). With switch (258) now pressed, processor (265) determines that trigger (226) is in the proximal-most position.

Should Front Seal Button toggle button (209) be in the “off” position at the stage depicted in FIG. 15C, processor (265) may automatically activate the electrodes of jaws (282, 284) in response to switch (258) being pressed. In other words, processor (265) may activate the electrodes without the user needing to further actuate activation button (230). By contrast, should Front Seal Button toggle button (209) be in the “on” position at the stage depicted in FIG. 15C, processor (265) may require the user to press activation button (230) to activate the electrodes of jaws (282, 284) after switch (258) has been pressed. In some such cases, regardless of whether Front Seal Button toggle button (209) is in the “on” position or the “off” position, the electrodes of jaws (282, 284) may remain energized so long as switch (258) is pressed or may be timed based on a predetermined threshold. As yet another variation, in some cases where Front Seal Button toggle button (209) is in the “off” position at the stage depicted in FIG. 15C, processor (265) may activate the electrodes of jaws (282, 284) in response to a footswitch or other external activation source being actuated.

In the state shown in FIG. 15D, trigger (226) has been returned to the distal-most position such that end effector (280) is open and no longer grasps tissue; and such that electrodes of jaws (282, 284) are no longer activated or energized. At this stage, the above process may be repeated at a different region of tissue. Alternatively, the user may select a different mode of operation for electrosurgical instrument (201) and use electrosurgical instrument (201) in the different selected mode. Alternatively, the process may be complete.

### IV. Examples of Combinations

The following examples relate to various non-exhaustive ways in which the teachings herein may be combined or applied. The following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

A surgical instrument, comprising: (a) a handle; (b) an end effector; (c) a trigger moveable relative to the handle between a first position and a second position, the trigger being further movable from the second position to a third position, the trigger being configured to actuate the end effector upon a movement of the trigger from the first position to the second position; (d) a lock configured to transition between a locked configuration and an unlocked configuration, the lock in the locked configuration being configured to prevent the trigger from moving from the second position to the first position, the lock in the unlocked configuration being configured to allow the trigger to transition from the second position to the first position; (e) a sensor, the trigger being configured to activate the sensor upon a movement of the trigger from the second position to the third position; and (f) a processor in electrical communication with the sensor and the lock, the processor being configured to: (i) receive a mode selection, the mode being selected from a plurality of available modes, the plurality of available modes including: (A) a latching mode, (B) a free movement mode, and (C) seal upon closure mode, (ii) detect when the trigger is in the third position via activation of the sensor by the trigger, and (iii) provide a response to detecting that the trigger is in the third position, the response being based on which mode from the plurality of available modes has been selected.

### EXAMPLE 2

The surgical instrument of Example 1, further comprising a user input in electrical communication with the processor, the user input being configured to: (i) receive the mode selection from the user, and (ii) transmit the mode selection from the user to the processor.

### EXAMPLE 3

The surgical instrument of Example 2, further comprising a monitor having a graphical user interface (GUI), the GUI providing the user input.

### EXAMPLE 4

The surgical instrument of any of Examples 1 through 3, the processor being further configured to activate the lock, to thereby prevent the trigger from moving from the second position to the first position, in response to detecting that the trigger is in the third position, if the latching mode was selected.

### EXAMPLE 5

The surgical instrument of Example 4, the processor being further configured to: (i) detect a subsequent activation of the sensor by the trigger, and (ii) deactivate the lock to thereby unlock the trigger from the second position, to thereby permit the trigger from moving from the second position to the first position, in response to detecting the subsequent activation of the sensor by the trigger, if the latching mode was selected.

### EXAMPLE 6

The surgical instrument of any of Examples 1 through 5, further comprising an activation button in electrical communication the processor, the activation button being operable to activate one or more electrodes of the end effector to thereby apply electrical energy to tissue.

### EXAMPLE 7

The surgical instrument of Example 6, the processor being further configured to refrain from activating the lock, and activate the electrodes, in response to: (A) detecting that the trigger is in the third position, and (B) activation of the activation button, a footswitch, or another external activation source, if the free movement mode was selected.

### EXAMPLE 8

The surgical instrument of any of Examples 6 through 7, the processor being further configured to refrain from activating the lock, and activate the electrodes, in response to activation of the activation button, if the free movement mode was selected.

### EXAMPLE 9

The surgical instrument of any of Examples 1 through 8, the processor being further configured to activate one or more electrodes of the end effector to thereby apply electrical energy to tissue, in response to detecting that the trigger is in the third position, if the seal upon closure mode was selected.

### EXAMPLE 10

The surgical instrument of any of Examples 1 through 9, the sensor comprising a switch.

### EXAMPLE 11

The surgical instrument of any of Examples 1 through 10, the lock including a resilient member configured to resiliently bias the lock toward the unlocked configuration.

### EXAMPLE 12

The surgical instrument of any of Examples 1 through 11, the lock including an actuator in electrical communication with the processor, the actuator being configured to actuate in response to a command from the processor, the actuator being configured to operably engage the trigger.

### EXAMPLE 13

The surgical instrument of any of Examples 1 through 12, the trigger including a trigger latch configured to engage the lock when the lock is in the locked configuration.

### EXAMPLE 14

The surgical instrument of any of Examples 1 through 13, the lock including a pivot arm having a lock pin, the lock pin being configured to engage the trigger to thereby maintain the trigger in the second position.

### EXAMPLE 15

The surgical instrument of any of Examples 1 through 14, the end effector including jaws with one or more electrodes operable to apply electrical energy to tissue.

### EXAMPLE 16

A surgical instrument, comprising: (a) a handle; (b) an end effector positioned distally in relation to the handle, the end effector comprising jaws and one or more electrodes; (c) a trigger operable to move from a first position to a second position to thereby drive the jaws from an open configuration to a closed configuration, the trigger being further operable to move from the second position to a third position; (d) a lock configured to lock the trigger within a range of movement; (e) a sensor configured to detect the trigger reaching the third position; (f) a user input configured to receive a mode selection from a user, the mode being selected from a plurality of available modes; and (g) a processor in electrical communication with the sensor, the lock, and the user input, the processor being configured to: (i) receive the mode selection from the user input to thereby transition to the mode selected from plurality of available modes, and (ii) either: (A) activate the lock in response to the sensor detecting the trigger reaching the third position, (B) refrain from activating the lock in response to the sensor detecting the trigger reaching the third position, or (C) activate the one or more electrodes to thereby apply electrical energy to tissue in response to detecting that the trigger is in the third position, based on which mode was selected from plurality of available modes.

### EXAMPLE 17

The surgical instrument of Example 16, further comprising a monitor having a graphical user interface (GUI), the user input comprising at least a portion of the GUI.

### EXAMPLE 18

The surgical instrument of any of Examples 16 through 17, further comprising an activation button, the activation button being operable to activate the one or more electrodes to thereby apply electrical energy to tissue.

### EXAMPLE 19

A method of using a surgical instrument, the surgical instrument including a handle, a trigger, an electrode activation button, a lock, and an end effector, the method including: (a) selecting a processor mode from a plurality of modes, the plurality of modes including: (i) a latching mode, (ii) a free movement mode, and (iii) seal upon closure mode; (b) moving the trigger relative to the handle from a first position to a second position, the end effector grasping tissue in response to the trigger being moved from the first position to the second position, the trigger activating a switch upon reaching the second position; and (c) in response to the trigger activating the switch, either: (i) activating the lock to thereby lock the trigger in the second position if the latching mode was selected, (ii) refraining from activating the lock, and activating the electrodes if the electrode activation button, a footswitch, or another external activation source is depressed, if the free mode was selected, or (iii) activating one or more electrodes of the end effector to apply energy to the grasped tissue if the seal upon closure mode was selected.

### EXAMPLE 20

The method of Example 19, the method further including using a graphical user interface (GUI) to: (i) provide selection of the mode, and (ii) indicate the mode selected.

### V. Miscellaneous

Any of the versions of the instruments described herein may include various other features in addition to or in lieu of those described above. While the examples herein are described mainly in the context of electrosurgical instruments, various teachings herein may be readily applied to a variety of other types of devices. By way of example only, the various teachings herein may be readily applied to other types of electrosurgical instruments, tissue graspers, tissue retrieval pouch deploying instruments, surgical staplers, surgical clip appliers, ultrasonic surgical instruments, etc. The teachings herein may be readily applied to any of the instruments described in any of the references cited herein. Any of the devices herein may also include one or more of the various features disclosed in any of the various references that are incorporated by reference herein. The teachings herein may thus be readily combined with the teachings of any of the references cited herein in numerous ways. Other types of instruments into which the teachings herein may be incorporated will be apparent to those skilled in the art in view of the teachings herein.

It should be appreciated that any patent, publication, or other disclosure material, in whole or in part, that is said to be incorporated by reference herein is incorporated herein only to the extent that the incorporated material does not conflict with existing definitions or other disclosure material set forth in this disclosure. The disclosure as explicitly set forth herein supersedes any conflicting material incorporated herein by reference. Any material, or portion thereof, that is said to be incorporated by reference herein, but which conflicts with existing definitions or other disclosure material set forth herein will only be incorporated to the extent that no conflict arises between that incorporated material and the existing disclosure.

The terms “proximal” and “distal” are defined herein relative to a surgeon, robotic arm, or other operator or structure grasping a surgical instrument having a distal surgical end effector. The term “proximal” refers the position of an element closer to the surgeon, robotic arm, or other operator or structure; and the term “distal” refers to the position of an element closer to the surgical end effector of the surgical instrument and further away from the surgeon or other operator or structure.

As used herein, an element or step recited in the singular and proceeded with the word “a” or “an” should be understood as not excluding plural of said elements or steps, unless such exclusion is explicitly stated. Furthermore, references to “one example” or “an example” are not intended to be interpreted as excluding the existence of additional examples that also incorporate the recited features. The use of “including,” “comprising,” or “having” and variations thereof herein is meant to encompass the items listed thereafter, and equivalents thereof, as well as additional items.

As used herein in the specification and claims, including as used in the examples and unless otherwise expressly specified, the terms “about” or “approximately” for any numerical values or ranges indicate a suitable dimensional tolerance, or other form of reasonable expected range, that allows the part or collection of components to function for its intended purpose as described herein. More specifically, “about” or “approximately” may refer to the range of values that are within ±10% of the recited value (e.g., “about 100” may refer to the range of values from 90 to 110, including 90, 110, 100, and all other values within the range of 90 and 110). Any numerical values given herein should also be understood to include about or approximately that value unless the context indicates otherwise. For example, if the value “10” is disclosed, then “about 10” is also disclosed. Any numerical range recited herein is intended to include all sub-ranges subsumed therein. The terms “approximately” and “about” are thus utilized herein to represent the degree by which a quantitative representation may vary from a stated reference without resulting in a change in the basic function of the subject matter at issue.

The term “substantially” is also utilized herein to represent the degree by which a quantitative representation may vary from a stated reference without resulting in a change in the basic function of the subject matter at issue. The term “substantially” shall therefore be understood to include a range of conditions or results that provide a functional equivalent to an explicitly stated condition or result. For instance, if a task is “substantially complete,” the result of the task having been substantially completed is functionally equivalent to the result that would have been achieved if the task had been perfectly completed. As another non-limiting example, a component that is “substantially straight” or “substantially flat,” an apparatus including a component that is “substantially straight” or “substantially flat” may provide a result or effect that is functionally equivalent to a result or effect that would be achieved by the same apparatus including the same component in a perfectly straight or perfectly flat configuration. The range implied by the term “substantially” should also be read to include the perfect result that is within that range. Thus, the term “substantially complete” shall be read as including “perfectly complete” while also including a range of completeness that is functionally equivalent to perfectly complete. As another example, terms such as “substantially straight” and “substantially flat” shall be read as including “perfectly straight” and “perfectly flat,” respectively; while also including a range of straightness or flatness that is functionally equivalent to perfectly straight or flat, respectively. As with the terms “approximately” and “about,” the term “substantially” may indicate a suitable dimensional tolerance, or other form of reasonable expected range, that allows a part or collection of components to function for its intended purpose as described herein.

The limitations of the following claims are not written in means-plus-function format and are not intended to be interpreted based on 35 U.S.C. § 112(f), unless such claim limitations expressly use the phrase “means for” followed by a statement of function void of further structure.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

I/We claim:

1. A surgical instrument, comprising:
(a) a handle;
(b) an end effector;
(c) a trigger moveable relative to the handle between a first position and a second position, the trigger being further movable from the second position to a third position, the trigger being configured to actuate the end effector upon a movement of the trigger from the first position to the second position;
(d) a lock configured to transition between a locked configuration and an unlocked configuration, the lock in the locked configuration being configured to prevent the trigger from moving from the second position to the first position, the lock in the unlocked configuration being configured to allow the trigger to transition from the second position to the first position;
(e) a sensor, the trigger being configured to activate the sensor upon a movement of the trigger from the second position to the third position; and
(f) a processor in electrical communication with the sensor and the lock, the processor being configured to:
(i) receive a mode selection, the mode being selected from a plurality of available modes, the plurality of available modes including:
(A) a latching mode,
(B) a free movement mode, and
(C) seal upon closure mode,
(ii) detect when the trigger is in the third position via activation of the sensor by the trigger, and
(iii) provide a response to detecting that the trigger is in the third position, the response being based on which mode from the plurality of available modes has been selected.

2. The surgical instrument of claim 1, further comprising a user input in electrical communication with the processor, the user input being configured to:
(i) receive the mode selection from the user, and
(ii) transmit the mode selection from the user to the processor.

3. The surgical instrument of claim 2, further comprising a monitor having a graphical user interface (GUI), the GUI providing the user input.

4. The surgical instrument of claim 1, the processor being further configured to activate the lock, to thereby prevent the trigger from moving from the second position to the first position, in response to detecting that the trigger is in the third position, if the latching mode was selected.

5. The surgical instrument of claim 4, the processor being further configured to:
(i) detect a subsequent activation of the sensor by the trigger, and
(ii) deactivate the lock to thereby unlock the trigger from the second position, to thereby permit the trigger from moving from the second position to the first position, in response to detecting the subsequent activation of the sensor by the trigger, if the latching mode was selected.

6. The surgical instrument of claim 1, further comprising an activation button in electrical communication the processor, the activation button being operable to activate one or more electrodes of the end effector to thereby apply electrical energy to tissue.

7. The surgical instrument of claim 6, the processor being further configured to refrain from activating the lock, and activate the electrodes, in response to:
(A) detecting that the trigger is in the third position, and
(B) activation of the activation button, a footswitch, or another external activation source,
if the free movement mode was selected.

8. The surgical instrument of claim 6, the processor being further configured to refrain from activating the lock, and activate the electrodes, in response to activation of the activation button, if the free movement mode was selected.

9. The surgical instrument of claim 1, the processor being further configured to activate one or more electrodes of the end effector to thereby apply electrical energy to tissue, in response to detecting that the trigger is in the third position, if the seal upon closure mode was selected.

10. The surgical instrument of claim 1, the sensor comprising a switch.

11. The surgical instrument of claim 1, the lock including a resilient member configured to resiliently bias the lock toward the unlocked configuration.

12. The surgical instrument of claim 1, the lock including an actuator in electrical communication with the processor, the actuator being configured to actuate in response to a command from the processor, the actuator being configured to operably engage the trigger.

13. The surgical instrument of claim 1, the trigger including a trigger latch configured to engage the lock when the lock is in the locked configuration.

14. The surgical instrument of claim 1, the lock including a pivot arm having a lock pin, the lock pin being configured to engage the trigger to thereby maintain the trigger in the second position.

15. The surgical instrument of claim 1, the end effector including jaws with one or more electrodes operable to apply electrical energy to tissue.

16. A surgical instrument, comprising:
(a) a handle;
(b) an end effector positioned distally in relation to the handle, the end effector comprising jaws and one or more electrodes;
(c) a trigger operable to move from a first position to a second position to thereby drive the jaws from an open configuration to a closed configuration, the trigger being further operable to move from the second position to a third position;
(d) a lock configured to lock the trigger within a range of movement;
(e) a sensor configured to detect the trigger reaching the third position;
(f) a user input configured to receive a mode selection from a user, the mode being selected from a plurality of available modes; and
(g) a processor in electrical communication with the sensor, the lock, and the user input, the processor being configured to:
(i) receive the mode selection from the user input to thereby transition to the mode selected from plurality of available modes, and
(ii) either:
(A) activate the lock in response to the sensor detecting the trigger reaching the third position,
(B) refrain from activating the lock in response to the sensor detecting the trigger reaching the third position, or
(C) activate the one or more electrodes to thereby apply electrical energy to tissue in response to detecting that the trigger is in the third position,
based on which mode was selected from plurality of available modes.

17. The surgical instrument of claim 16, further comprising a monitor having a graphical user interface (GUI), the user input comprising at least a portion of the GUI.

18. The surgical instrument of claim 16, further comprising an activation button, the activation button being operable to activate the one or more electrodes to thereby apply electrical energy to tissue.

19. A method of using a surgical instrument, the surgical instrument including a handle, a trigger, an electrode activation button, a lock, and an end effector, the method including:
(a) selecting a processor mode from a plurality of modes, the plurality of modes including:
(i) a latching mode,
(ii) a free movement mode, and
(iii) seal upon closure mode;
(b) moving the trigger relative to the handle from a first position to a second position, the end effector grasping tissue in response to the trigger being moved from the first position to the second position, the trigger activating a switch upon reaching the second position; and
(c) in response to the trigger activating the switch, either:
(i) activating the lock to thereby lock the trigger in the second position if the latching mode was selected,
(ii) refraining from activating the lock, and activating the electrodes if the electrode activation button, a footswitch, or another external activation source is depressed, if the free mode was selected, or
(iii) activating one or more electrodes of the end effector to apply energy to the grasped tissue if the seal upon closure mode was selected.

20. The method of claim 19, the method further including using a graphical user interface (GUI) to:
(i) provide selection of the mode, and
(ii) indicate the mode selected.
